(19) United States
(12) Patent Application Publication
(10) Pub. No.: US 2004/0157931 A1
(43) Pub. Date: Aug. 12, 2004

(54) Acid disinfectant composition comprising an anion surfacant

(76) Inventors: Jeong Chan Ra, Suwon Kyungki-do (KR); Kyung Sun Kang, Suwon Kyungki-do (KR); Yong Ho Park, Seoul (KR); Yong Soon Lee, Kwacheon-si Kyungki-do (KR); Hae Jung Han, Yongin-si Kyungki-do (KR); Jong Eun Lee, Daejeon (KR)

Correspondence Address: David A Einhorn, Anderson Kill & Olick, 1251 Avenue of the Americas, New York, NY, 10020, US

(21) Appl. No.: 10/483,172

(22) PCT Filed: Jul. 6, 2002
(86) PCT No.: PCT/KR02/01283

(30) Foreign Application Priority Data
Jul. 6, 2001 (KR) 2001/40257
May 21, 2002 (KR) 2002/28189

(51) Int. Cl. A01N 37/00; A61K 31/185

(52) U.S. Cl. 514/576

(57) ABSTRACT

The present invention relates to an improved acid disinfectant composition which exhibits excellent bactericidal activity against a wide spectrum of Gram-negative and Gram-positive bacteria.

### FIELD OF THE INVENTION

The present invention relates to an improved acid disinfectant composition which comprises an anion surfactant.

### BACKGROUND OF THE INVENTION

Various disinfection formulations have been developed to protect people as well as domestic animals from various pathogens, but acid formulations are considered to be much safer to use than others. Such acid formulations have been prepared using non-ionic surfactants, and citric acid formulations have been widely used to control such pathogens as Clostridium sporogenes (M. H. Silla Santos and J. Torres Zarzo, Int. J. Food Microbiology, 29, 241-254, 1996), Listeria monocytogenes (K. M. Young and Peggy M. Foefeding, J. Appl. Bacteriology, 74, 515-520, 1993), Salmonella spp. (E. T. Barnhart et al., Poultry Sci., 7,8 32-37, 1999), E. coli (J. Appl. Microbiol., 90 771-778, 2001) and FMD virus (Rev. Sci. Tech., 9, 1139-1155, 1990).

However, a conventional acid formulation is not satisfactorily effective against some pathogens, e.g., E. coli, and Gram-positive bacteria such as S. aureus and L. monocytogenes, and its disinfectant activity becomes poor in the presence of organic matters. Therefore, the present inventors have endeavored to develop an improved disinfectant composition that has high bactericidal activity against a wide spectrum of bacteria even in the presence of organic matters.

### SUMMARY OF THE INVENTION

Accordingly, it is an object of the present invention to provide an acid disinfectant composition which has higher bactericidal activity than conventional formulations, particularly in the presence of an organic matter.

In accordance with one aspect of the present invention, there is provided an aqueous disinfectant composition comprising an acid and an anion surfactant, the amount of the anion surfactant being 1 to 50 parts by weight based on 100 parts by weight of the acid.

### DETAILED DESCRIPTION OF THE INVENTION

The anion surfactant of the disinfectant composition of the present invention, which enhances disinfectant activity against Gram-negative and Gram-positive bacteria, is selected from the group consisting of sodium dodecyl sulfate, alkyl benzene sulfonic acids, alkyl and alkyl ether sulfates, alkyl sulfosuccinates, alkyl phosphates, amino acid derivatives and a mixture thereof, preferably sodium dodecyl sulfate, linear alkylbenzene sulfonic acid, ammonium lauryl sulfate, sodium dioctyl sulfosuccinate, mono lauroyl phosphate, tetraethylammonium (TEA) lauryl sarcosinate and a mixture thereof.

The acid of the inventive disinfectant composition is selected from the group consisting of citric acid, acetic acid, hydrochloric acid, sodium cyanate, peracetic acid and a mixture thereof.

The disinfectant composition of the present invention may further comprise one or more additional organic acids selected from the group consisting of malic acid, lactic acid, tartaric acid and succinic acid, the amount of the additional organic acid being preferably 1 to 50 parts by weight based on 100 parts by weight of the acid.

Also, the disinfectant composition of the present invention may further comprise a pH lowering agent (e.g., sulfamic acid), an oxidizing agent (e.g., potassium persulfate), a pH buffering agent (e.g., sodium hexametaphosphate), or an antiseptic (e.g., zinc sulfate).

The disinfectant composition of the present invention may be prepared simply by dissolving the above-mentioned ingredients in water to obtain an aqueous disinfectant composition. The composition may be diluted to any desired concentration by adding water thereto.

The inventive disinfectant composition prepared as above exhibits strong bactericidal activity against a wide spectrum of Gram-negative and Gram-positive bacteria, even in the presence of an organic matter.

The following Examples are included to farther illustrate the present invention without limiting its scope.

### EXAMPLE 1

### Measurement of Bactericidal Activities be Anion Surfactant

Step 1: Culture of Bacterial Strains

The following strains, which are generally known to cause animal diseases, were used in disinfectant activity measurements: Salmonella cholerasuis (diarrhea and respiratory diseases, ATCC 7001), Escherichia coli O157:H7 (food poisoning, ATCC 43894), Streptococcus uberis (mastitis, ATCC 27958), Pasteurella haemolytica (pneumonia and respiratory diseases), Staphylococcus aureus (mastitis and food poisoning), Listeria monocytogenes (food poisoning and abortion), Bordetella bronchiseptica (contagious bronchitis), Streptococcus agalactiae (mastitis) and Saccharomyces cerevisiae (mastitis). Pasteurella haemolytica, Staphylococcus aureus, Listeria monocytogenes, Bordetella bronchiseptica, Streptococcus agalactiae and Saccharomyces cerevisiae were kindly provided by laboratory of Veterinary Microbiology, Veterinary Medicine, College of Veterinary Medicine, Seoul National University.

Each strain was incubated in a blood media (agar 15 g, beef extract 3 g, peptone 5 g, sheep blood 40 ml) at 25° C. for 24 hours to acquire monocolonies. Saccharomyces cerevisiae was inoculated in Sabouraud media (bacto-neopeptone 10 g, bacto-dextrose 40 g, bacto-agar 15 g) and the other strains, in TSB media (tryptic soy broth; bacto-typtone 17 g, bacto-soytone 3 g, sodium chloride 5 g, dipotassium phosphate 2.5 g), followed by incubating at 25° C. for 24 hours.

Step 2: Preparation of Disinfectant Compositions

Four disinfectant compositions shown in Table 1 were prepared: Formulations 1 and 2 each comprised a non-ionic surfactant, poloxamer 188 (Basf); Formulation 4 contained an anion surfactant, sodium dodecyl sulfate (DUKSAN PURE CHEMICAL CO. LTD); and Formulation 3 was a control which had the same composition as Formulation 4 except for the absence of the surfactant.

TABLE 1

| | Composition |
|---|---|
| Formulation 1 | Citric acid 5 g, malic acid 435 g, poloxamer 188 5 g |
| Formulation 2 | Citric acid 90 g, malic acid 90 g, |
| | sulfamic acid 20 g, potassium |
| | persulfate 50 g, zinc sulfate 100 g, poloxamer 188 5 g |
| Formulation 3 | Citric acid 100 g, malic acid 100 g, |
| | sulfamic acid 100 g, potassium |
| | persulfate 150 g, sodium hexametaphosphate 100 g |
| Formulation 4 | Formulation 3 + sodium dodecyl sulfate 50 g |

Each of the compositions was dissolved in water to a concentration of 1 g/10 ml and serially diluted with water. The pH values of 1,000 time diluted solutions of Formulations 1, 2, 3 and 4 were 3.35, 3.55, 3.41 and 3.32, respectively.

Step 3: Bactericidal Activities of Disinfectant Compositions

2.5 mi of a bacterial cell cultured in TBS was mixed with 2.5 ml of a disinfectant solution of variable dilution to obtain a cell suspension having a cell concentration of 10⁷ CFU/ml, and the suspension was cultured at 37° C. for 30 minutes. A loop of the cultured cell suspension was added to 10 ml of MHB (Meuller Hinton Broth; beef infusion form 300 g, bacto-casamino acid 17.5 g, bacto-soluble starch 1.5 g) and serially diluted until the pH of the diluted solution became 7.3 to stop the action of the acid disinfectant.

MIC (minimum inhibitory concentration) was defined as the minimum concentration of the disinfectant to inhibit the growth of the bacterial cells in the above-mentioned suspension (MIC suspension). The fact that bacteria in each MIC suspension were indeed eradicated was confirmed by observing no bacterial growth when a portion of the MIC suspension was sub-cultured in a blood media at 37° C. for 36 hours. The results are shown in Table 2, wherein the values of MIC are expressed in multiples of dilution.

TABLE 2

MIC (multiples of dilution)

| Strains | F 1 | F 2 | F 3 | F 4 |
|---|---|---|---|---|
| Salmonella cholerasuis | 400 | 400 | 640 | 1280 |
| Staphylococcus aureus | N.D | N.D | 80 | 640 |
| Pasteurella haemolytica | — | — | 2560 | 5120 |
| Bordetella bronchiseptica | 400 | 200 | 20 | 1280 |
| Escherichia coli O157:H7 | 20 | 20 | 40 | 640 |
| Streptococcus uberis | — | — | 1600 | 3200 |
| Listeria monocytogenes | N.D | N.D | 40 | 1280 |
| Streptococcus agalactiae | 80 | 80 | — | — |

N.D: not determined
F: formulation

As can be seen in Table 2, the disinfectant compositions 1 and 2 were not effective against E. coli and Gram-positive strains (S. aureus and L. monocytogenes). In contrast, the disinfectant composition of the present invention comprising sodium dodecyl sulfate (Formulation 4) had excellent bactericidal activities against all of the strains examined.

### EXAMPLE 2

### Bactericidal Activities of Disinfectant Composition Containing Barious Anion Surfactants Measured in the Presence of an Organic Matter (Milk)

The procedure of Example 1 was repeated with a suspension containing 2 wt % of milk using a formulation equivalent to Formulation 4 but containing, in place of sodium dodecyl sulfate, a) linear alkylbenzene sulfonic acid (LAS, Miwon Commercial Co. Ltd), an alkyl benzene sulfonic acid, b) ammonium lauryl sulfate (MICOLIN A-428, Miwon Commercial Co. Ltd), an alkyl and alkyl ether sulfate, c) sodium dioctyl sulfosuccinate (MICONATE DOS, Miwon Commercial Co. Ltd), an allyl sulfosuccinate, d) mono lauroyl phosphate (MIPHOS ML, Miwon Commercial Co. Ltd), an alkyl phosphate, or e) TEA lauryl sarcosinate (IAMI TL-30, Miwon Commercial Co. Ltd), an amino acid derivative. The results are shown in Table 3.

TABLE 3

| | MIC (multiples of dilution) | | | | |
|---|---|---|---|---|---|
| Strains | a | b | c | d | e |
| Salmonella cholerasuis | 1280 | 640 | 640 | 320 | 640 |
| Staphylococcus aureus | 640 | 1280 | 640 | 320 | 1280 |
| Pasteurella haemolytica | 3200 | 1280 | 3200 | 640 | 1280 |
| Bordetella bronchiseptica | 1280 | 640 | 640 | 320 | 320 |
| Escherichia coli O157:H7 | 640 | 320 | 640 | 640 | 1280 |
| Streptococcus uberis | 3200 | 2560 | 1280 | 2560 | 640 |
| Listeria monocytogenes | 3200 | 1280 | 1280 | 640 | 640 |

As can be seen in Table 3, the disinfectant compositions of the present invention comprising various anion surfactants show excellent disinfectant activities against Gram-negative and Gram-positive bacteria, even in the presence of milk, an organic matter.

What is claimed is:

1. An aqueous disinfectant composition comprising an acid and an anion surfactant, the amount of the anion surfactant being 1 to 50 parts by weight based on 100 parts by weight of the acid.

2. The composition of claim 1, wherein the anion surfactant is selected from the group consisting of sodium dodecyl sulfate, an alkyl benzene sulfonic acid, an alkyl and alkyl ether sulfate, an alkyl sulfosuccinate, an alkyl phosphate, an amino acid derivative and a mixture thereof.

3. The composition of claim 2, wherein the anion surfactant is selected from the group consisting of sodium dodecyl sulfate, linear alkylbenzene sulfonic acid, ammonium lauryl sulfate, sodium dioctyl sulfosuccinate, mono lauroyl phosphate, tetraethylammonium lauryl sarcosinate and a mixture thereof.

4. The composition of claim 1, wherein the acid is selected from the group consisting of citric acid, acetic acid, hydrochloric acid, sodium cyanate, peracetic acid and a mixture thereof.

5. The composition of claim 1, which further comprises one or more organic acids selected from the group consisting of malic acid, lactic acid, tartaric acid and succinic acid, in an amount ranging from 1 to 50 parts by weight based on 100 parts by weight of the acid.
